(19) United States
(12) Patent Application Publication
(10) Pub. No.: US 2025/0104231 A1
(43) Pub. Date: Mar. 27, 2025

(54) DORSAL MEDULLA SURFACE TEXTURE AS AN IMAGING METRIC TO DISTINGUISH BETWEEN NEUROLOGICAL DISORDERS SYSTEMS AND METHODS

(71) Applicant: The Board of Regents of the University of Texas System, Austin, TX (US)

(72) Inventor: Darin T. Okuda, Dallas, TX (US)

(21) Appl. No.: 18/833,074

(22) PCT Filed: Feb. 23, 2023
(86) PCT No.: PCT/US23/63145

§ 371 Date: Jul. 25, 2024

Related U.S. Application Data
(60) Provisional application No. 63/314,308, filed on Feb. 25, 2022.

(51) Int. Cl. G06T 7/00; G06T 7/64; G16H 30/40; G16H 50/20

(52) U.S. Cl. CPC G06T 7/0012; G06T 7/64; G16H 30/40; G16H 50/20; G06T 2207/10081; G06T 2207/30016

(57) ABSTRACT

Systems and methods to determine an increased likelihood of a neurological disorder based on a dorsal surface texture of the medulla oblongata. The method includes selecting, using a computational topography software, a region of interest (ROI) within a diameter ring at a lower dorsal posterior medulla encompassing a region of a clava. The method includes analyzing, using the computational topography software, surface complexity of the ROI using a metric maximum curvature analysis that provides a local maximum curvature on discretized triangular mesh surface representations. The method includes determining between introverted triangles and extroverted triangles and calculating a first number of the introverted triangles and a second number of the extroverted triangles in the ROI. The method also includes the determination of the presence or absence of a distinct spatial dissemination pattern of introverted triangles extending craniocaudally within a center of the ROI.

### CROSS-REFERENCE TO RELATED APPLICATION

This application claims priority to U.S. Provisional Application No. 63/314,308 filed Feb. 25, 2022, which is incorporated by reference herein in its entirety.

### BACKGROUND

### 1. Field

The present inventive concept relates in general to the field of neuroimmunology imaging and, in particular, systems and methods to use a dorsal medulla surface texture as an imaging metric for neurological disorders, such as neuromyelitis optica spectrum disorder (NMOSD).

### 2. Discussion of Related Art

The accurate and timely diagnosis of rare neurological conditions is critical to prescribing the appropriate FDA-approved treatments to reduce the risk for acute inflammatory exacerbations and permanent disability. Within the field of neuroimmunology, distinct disorders including neuromyelitis optica spectrum disorder (NMOSD), multiple sclerosis (MS), and myelin oligodendrocyte glycoprotein associated disorders (MOGAD), at times, demonstrate overlapping clinical symptoms, cerebrospinal fluid profiles, antibody profile data, and findings on magnetic resonance imaging (MRI). The accurate diagnosis of such patients remains challenging, even in the setting of established criteria, and many patients early in the disease course may not meet the criteria for a specific condition.

Therefore, there is a need in the art for a system and method for determining conformational differences between neurological disorders, such as between AQP4-IgG positive and AQP4-IgG negative NMOSD, MOGAD, and MS patients based on neuroimaging measure of quantifications.

### SUMMARY

To address the above-noted problems, systems and methods are disclosed herein for determining an increased likelihood of a neurological disorder based on a dorsal surface texture of a medulla oblongata. According to some examples, the method includes selecting, using a computational topography software, a region of interest (ROI) within a diameter ring at a lower dorsal posterior medulla encompassing a region of a clava. According to some examples, the method includes analyzing, using the computational topography software, surface complexity of the ROI using a metric maximum curvature analysis that provides a local maximum curvature on discretized triangular mesh surface representations. According to some examples, the method includes determining between introverted triangles and extroverted triangles and calculating a first number of the introverted triangles and a second number of the extroverted triangles in the ROI.

For example, a system selects, uses a computational topography software, a region of interest (ROI) within a diameter ring at a lower dorsal posterior medulla encompassing the region of the clava. According to some examples, the system analyzes, uses the computational topography software, surface complexity of the ROI using a metric maximum curvature analysis that provides a local maximum curvature on discretized triangular mesh surface representations. According to some examples, the system determines between introverted triangles and extroverted triangles and calculates a first number of the introverted triangles and a second number of the extroverted triangles in the ROI.

In another example, a system for determining an increased likelihood of a neurological disorder based on a dorsal surface texture of a medulla oblongata is provided that includes a storage (e.g., a memory configured to store data, such as virtual content data, one or more images, etc.) and one or more processors (e.g., implemented in circuitry) coupled to the memory and configured to execute instructions and, in conjunction with various components (e.g., a network interface, a display, an output device, etc.), cause the system to select, use a computational topography software, a region of interest (ROI) within a diameter ring at a lower dorsal posterior medulla encompassing the region of the clava; analyze, use the computational topography software, surface complexity of the ROI using a metric maximum curvature analysis that provides a local maximum curvature on discretized triangular mesh surface representations; and determine between introverted triangles and extroverted triangles and calculate a first number of the introverted triangles and a second number of the extroverted triangles in the ROI.

### BRIEF DESCRIPTION OF THE DRAWINGS

To describe the manner in which the above-recited and other advantages and features of the disclosure can be obtained, a more particular description of the principles briefly described above will be rendered by reference to specific embodiments thereof which are illustrated in the appended drawings. Understanding that these drawings depict only exemplary embodiments of the disclosure and are not therefore to be considered to be limiting of its scope, the principles herein are described and explained with additional specificity and detail through the use of the accompanying drawings in which:

FIGS. 1A-1C illustrate examples method of determining an increased likelihood of a neurological disorder based on a dorsal surface texture of a medulla oblongata, in accordance with some examples;

FIG. 2 illustrates a region of interest at the dorsal medulla (yellow circle) along with the corresponding location (white mesh) within 3-dimensional (3D) mesh models (blue) and 3D visualization models containing the region of interest demonstrating the associated planar contours and surface topography, in accordance with some examples;

FIG. 3A illustrates texture characteristics within the region of interest at the dorsal medulla from patients with NMOSD, MS, and MOGAD, demonstrating differences in the number and pattern of triangles with introverted (blue) and extroverted (red) planar contours, in accordance with some examples;

FIG. 3B illustrates an example three-dimensional image of the dorsal aspect of the brainstem and upper cervical spinal cord created using data from two MRI time points;

FIG. 4 illustrates plots for the number of triangles representing introverted (A) and extroverted (B) planar contours measured cross-sectionally by group, in accordance with some examples;

FIG. 5 illustrates a table for baseline demographic and clinical characteristics of the study cohorts, in accordance with some examples; and

FIG. 6 shows an example of a computing system in accordance with some aspects of the present disclosure.

### DETAILED DESCRIPTION

Various embodiments of the disclosure are discussed in detail hereafter. While specific implementations are discussed, it should be understood that this is done for illustration purposes only. A person skilled in the relevant art will recognize that other components and configurations may be used without parting from the spirit and scope of the disclosure.

Additional features and advantages of the disclosure will be set forth in the description which follows, and in part will be obvious from the description or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims or can be learned by the practice of the principles set forth herein.

The disclosed examples are directed to determining an increased likelihood of a neurological disorder based on a dorsal surface texture of a medulla oblongata. The accurate and timely diagnosis of rare neurological conditions is critical to allow for the prescription of the appropriate FDA-approved treatments that reduce the risk for acute inflammatory exacerbations and permanent disability. Within the field of neuroimmunology, distinct disorders including neuromyelitis optica spectrum disorder (NMOSD), multiple sclerosis (MS), and myelin oligodendrocyte glycoprotein associated disorders (MOGAD), at times, demonstrate overlapping clinical symptoms, cerebrospinal fluid profiles, antibody profile data, and findings on magnetic resonance imaging (MRI). Thus, the accurate diagnosis of such patients remains challenging, even in the setting of established criteria, and many patients early in the disease course may not meet the criteria for a specific condition.

Remarkable autoimmune events localized at optic nerves more proximal to the chiasm, longitudinally extensive lesions within the spinal cord, and clinical experiences of hiccups or protracted nausea and vomiting resulting from aquaporin 4-IgG (AQP4) injury at the area postrema or upper cervical spinal cord typifies clinical and radiological characteristics of NMOSD. Unilateral or bilateral involvement of the area postrema along with the medullary floor of the fourth ventricle was demonstrated histopathologically in patients with NMOSD and found to be unique when compared to MS patients and control subjects. Although a greater increase in risk is present with involvement of the area postrema and surrounding anatomical regions, not all patients, even those who are AQP4-IgG positive experience associated symptoms suggesting tissue involvement in the absence of clinical symptomatology.

Therefore, there is a need in the art to improve clinical characterization, such as by evaluating 3-dimensional magnetic resonance imaging (MRI) conformational differences in dorsal medulla texture to determine between certain neurological disorders and likelihoods thereof based on differences in a neuroimaging measure of the dorsal medulla texture.

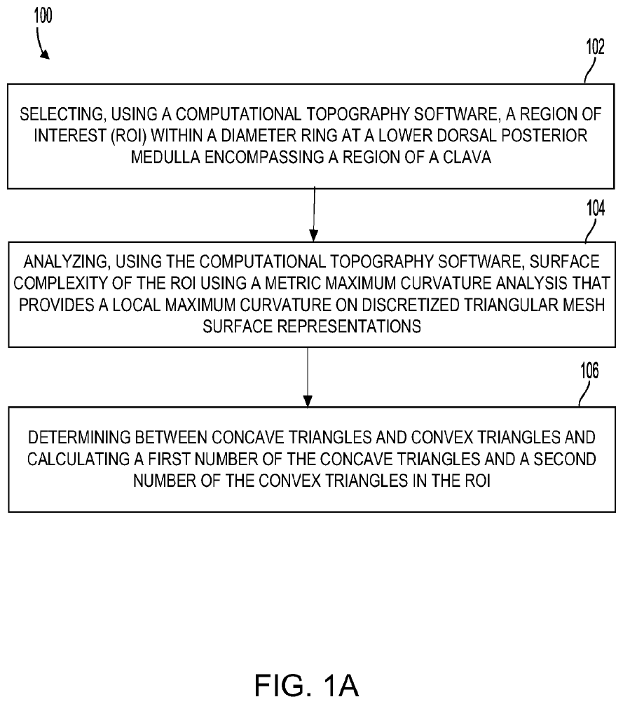

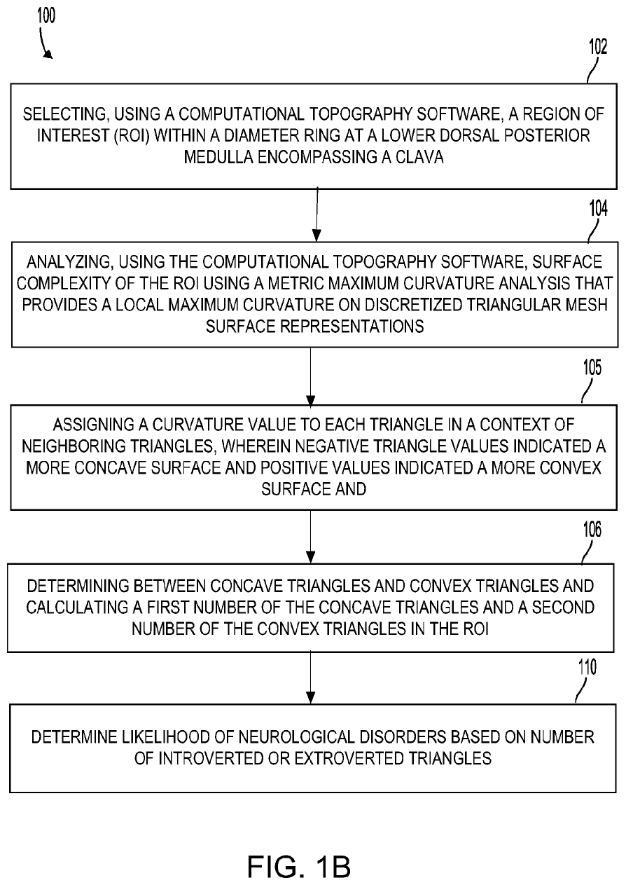

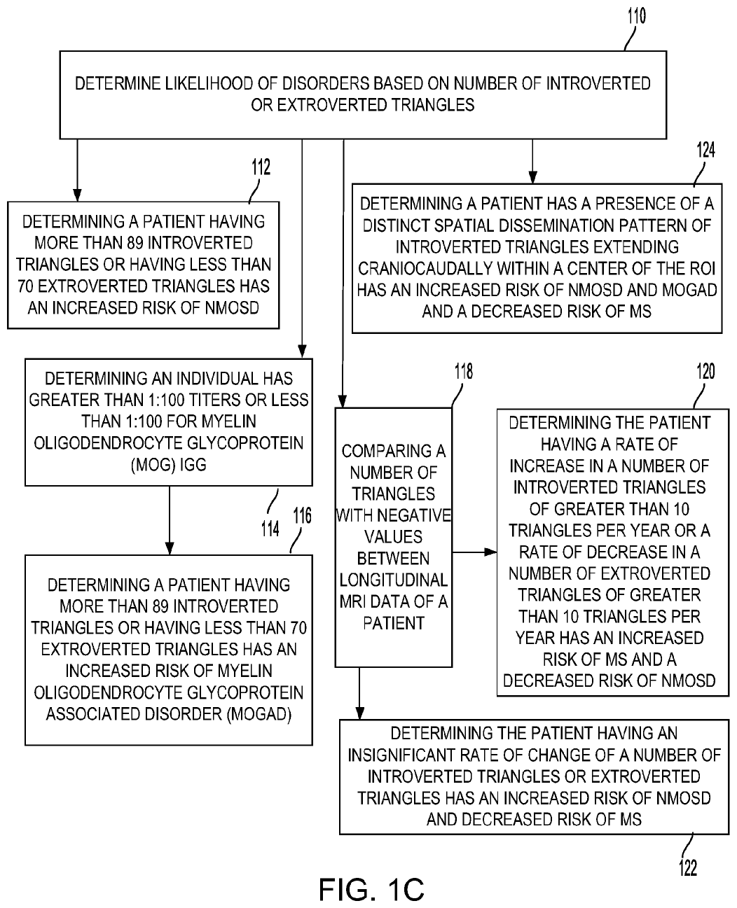

FIGS. 1A-1C illustrate example methods 100A-100C of determining an increased likelihood of a neurological disorder based on a dorsal surface texture of a medulla oblongata, in accordance with some examples. Although the example methods 100A-100C depict a particular sequence of operations, the sequence may be altered without departing from the scope of the present disclosure. For example, some of the operations depicted may be performed in parallel or in a different sequence that does not materially affect the function of the method 100. In other examples, different components of an example device or system that implements the method 100 may perform functions at substantially the same time or in a specific sequence.

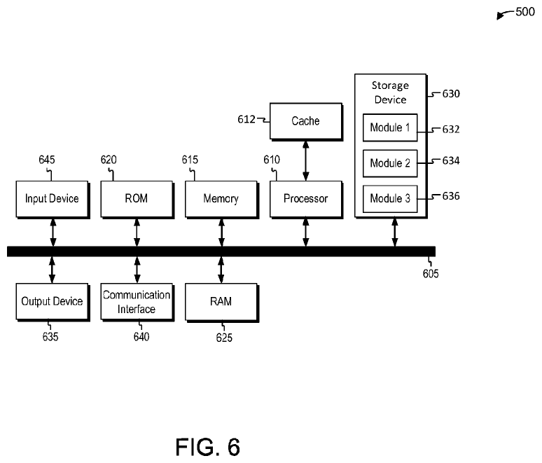

According to some aspects, the method includes using non-registered (cross-sectional measures in single MRI time point) or registered (the alignment of images from more than one MRI time point to ensure spatial correspondence of anatomy and image intensities) 3D T1-weighted magnetic resonance imaging (MRI) sequences for understanding features that inform on tissue integrity at the time of a patient's first presentation and as a means to identify shape changes of structures relative to previous MRI data, respectively. With respect to the cross-sectional measures in single MRI time point, to identify such a signal that demonstrates a high degree of accuracy for the diagnosis of NMOSD relative to MS on a single scan is remarkable. In another embodiment, texture results from multiple single MRI studies performed at different medical institutions may be used to further test for the diagnosis of NMOSD versus MS or to evaluate disease behavior in the presence or absence of treatment. The technique of registration is used when there is more than one MRI time point typically from MRI images originating from the same MRI unit. This allows for normalization of data between studies for more accurate measures. For example, the processor 610 illustrated in FIG. 6 may use the non-registered or registered 3D T1-weighted magnetic resonance imaging (MRI) sequences for track position and shape changes of structures. The method may include identifying the ROI from a superior colliculus of a midbrain to a caudal end of the medulla oblongata from non-contrast-enhanced 3D isotropic T1-weighted magnetic resonance imaging (MRI) sequences. For example, the processor 610 illustrated in FIG. 6 may identify the ROI from a superior colliculus of a midbrain to a caudal end of the medulla oblongata from non-contrast-enhanced 3D isotropic T1-weighted magnetic resonance imaging (MRI) sequences.

The method may further include a research phase of comparing a number of triangles with negative values within ROIs of a plurality of patients. For example, the processor 610 illustrated in FIG. 6 may compare a number of triangles with negative values within ROIs of a plurality of patients. In some aspects, a higher number of triangles with negative values informed on more introverted features within the region of interest.

According to some aspects, the method includes selecting, using a computational topography software, a region of interest (ROI) within a diameter ring at a lower dorsal posterior medulla encompassing the region of the clava at step 102. For example, the processor 610 illustrated in FIG. 6 may select, use a computational topography software, a region of interest (ROI) within a diameter ring at a lower dorsal posterior medulla encompassing the region of the clava.

The ROI may have a craniocaudal dimension of 50 mm from the superior colliculus of the midbrain to the caudal end of the medulla from non-contrast enhanced 3D isotropic T1-weighted sequences identified within the field of view from a brain MRI study. The brain MRI study may be isolated using Materialise Mimics (version 22.0; Materialise NV, Leuven, Belgium) and image masks were generated from both MRI time points. The ROI was strategically selected due to consistent anatomical boundaries amongst patients and the lack of impact on this structure with varying head positions.

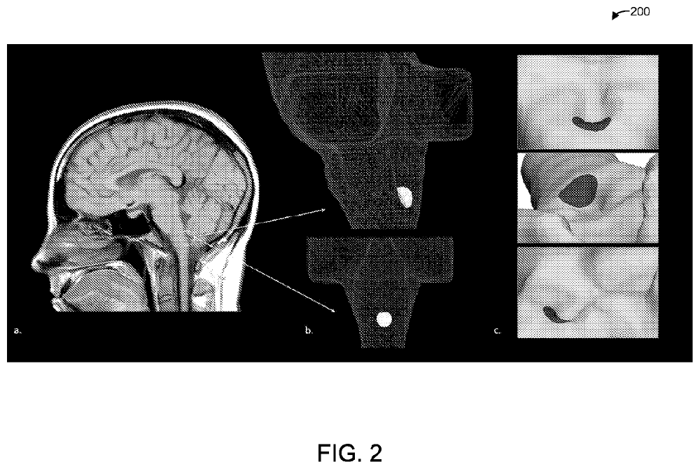

Surface complexity was computed by selecting a 5 mm diameter ring at the lower dorsal posterior medulla encompassing the region of the clava (see FIG. 2). Within the specified region of interest, surface complexity may be analyzed using maximum curvature analysis, a metric providing the local maximum curvature on discretized triangular mesh surface representations, using proprietary in-house developed computational topography software.

Before an analysis, an operation to unify triangle size may be implemented. A least-squares fitting technique encompassing computational measures at each triangle node may be used to compute a curvature measure for every triangle within the region of interest. This allowed for quantification of how the local surface deviates from a flat plane or local surface bending through the study of two orthogonal principal directions on its tangent plane.

Therefore, according to some aspects, the method includes computing, using the computational topography software, a curative measure for triangles within the ROI using a least-square fitting technique encompassing computational measure at each triangle node to unify triangle size. For example, the processor 610 illustrated in FIG. 6 may compute, use the computational topography software, a curative measure for triangles within the ROI using a least-square fitting technique encompassing computational measure at each triangle node to unify triangle size.

According to some aspects, the method includes analyzing, using the computational topography software, surface complexity of the ROI using a metric maximum curvature analysis that provides a local maximum curvature on discretized triangular mesh surface representations at step 104. For example, the processor 610 illustrated in FIG. 6 may analyze, use the computational topography software, surface complexity of the ROI using a metric maximum curvature analysis that provides a local maximum curvature on discretized triangular mesh surface representations.

In another example, as shown in FIG. 1B, the determining between the introverted triangles and the extroverted triangles further comprises assigning a curvature value to each triangle in a context of neighboring triangles in step 105. For example, the processor 610 illustrated in FIG. 6 may assign a curvature value to each triangle in a context of neighboring triangles. In some aspects, negative triangle values indicated a more introverted surface and positive values indicated a more extroverted surface.

By assigning a curvature value to each triangle in the context of neighboring triangles, the local curvature within the ROI may be utilized as an indicator of the change in surface complexity. Negative triangle values indicated a more introverted surface and positive values reflected more extroverted features. A higher number of triangles with negative values informed on more introverted features within the region of interest. The number of triangles with negative values within the region of interest was then compared between NMOSD, MS patients, and other groups.

According to some aspects, the method includes determining between introverted triangles and extroverted triangles and calculating a first number of the introverted triangles and a second number of the extroverted triangles in the ROI at step 106. For example, the processor 610 illustrated in FIG. 6 may determine between introverted triangles and extroverted triangles and calculate a first number of the introverted triangles and a second number of the extroverted triangles in the ROI.

According to some aspects, as shown in FIG. 1B, based on the introverted triangles and extroverted triangles, the method includes determining a likelihood of certain neurological disorders in step 110, which continues onto FIG. 1C.

For example, the method includes determining a patient having more than 89 introverted triangles or having less than 70 extroverted triangles has an increased risk of NMOSD at step 112. For example, the processor 610 illustrated in FIG. 6 may determine a patient having more than 89 introverted triangles or have less than 70 extroverted triangles has an increased risk of NMOSD. Furthermore, the method includes determining an individual has greater than 1:100 titers or less than 1:100 for myelin oligodendrocyte glycoprotein (MOG) IgG at step 114. For example, the processor 610 illustrated in FIG. 6 may determine an individual has greater than 1:100 titers or less than 1:100 for MOG IgG. According to some aspects, the method includes determining a patient having more than 89 introverted triangles or having less than 70 extroverted triangles has an increased risk of MOGAD in step 116. For example, the processor 610 illustrated in FIG. 6 may determine a patient having more than 89 introverted triangles or having fewer than 70 extroverted triangles has an increased risk of MOGAD.

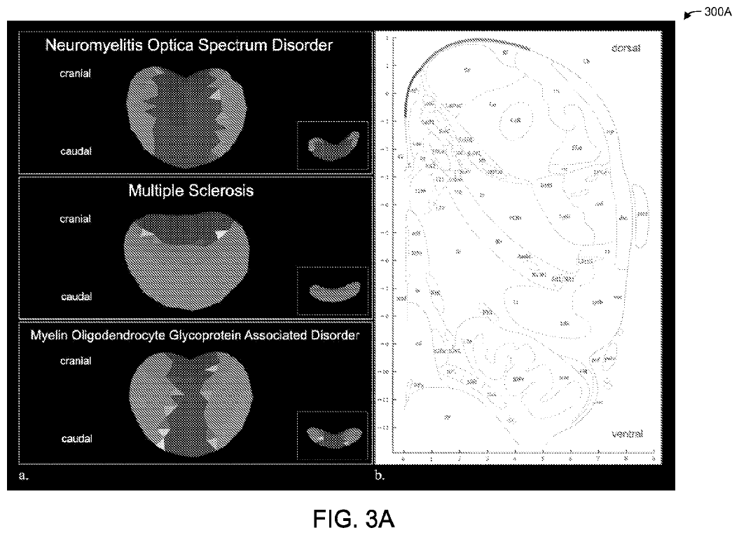

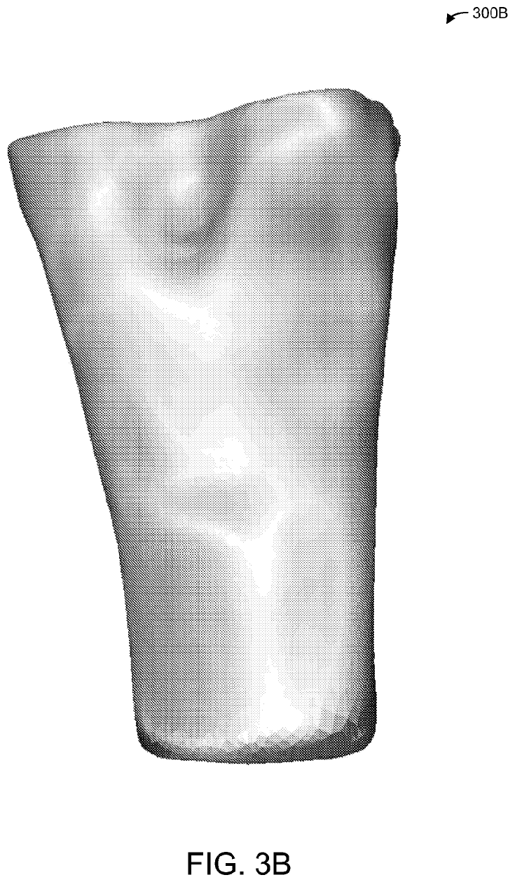

Such a determination is based on the research that resulted in the present diagnostic technology. The research found that a significantly higher median number of introverted triangle counts, representing more complex and introverted tissue topology, was observed in the NMOSD (median (IQR); 100.5 (23.5)) cohort when compared to MS (65 (20.25)) (p<0.0001) in. No difference in cross-sectional values was observed when comparing NMOSD and MOGAD (p=0.1), AQP4-IgG positive versus AQP4-IgG negative NMOSD (p=0.66), MOGAD High-Titer (HT) versus MOGAD Low-Titer (LT) (p=0.65), and no difference between MS and the combined group of other neuroimmunological conditions (p=0.57) (see FIG. 3).

The research found that the ideal cutoff for triangles representing an introverted planar contour consistent with introverted characteristics was identified at >89 with an in-sample AUC of 0.945 obtained (95% CI=[0.884, 1.006]. Based on this model, the following were obtained for the identification of NMOSD: sensitivity=0.77, specificity=1, PPV=1, and NPV 0.85 (out-of-sample sensitivity=0.83, specificity=1, PPV=1, and NPV=0.85).

Furthermore, a significantly lower median number of triangles representing an extroverted planar contour was identified between NMOSD (58 (24.25)) and MS patients (88 (19)) (p<0.0001), meaning more extroverted topographical characteristics were present than introverted. Patients with MOGAD also were identified with a significantly lower median number of triangles when compared to MS (p=0.0002) but not with NMOSD (p=0.30). No difference was observed when comparing AQP4-IgG positive versus AQP4-IgG negative NMOSD (p=0.27), MOGAD HT versus MOGAD LT (p=0.51), and no difference between MS and the combined group of other neuroimmunological conditions (p=0.70).

The research found that the ideal cutoff for level triangles representing an extroverted planar contour consistent with extroverted characteristics was identified at <70 with an in-sample AUC of 0.858 obtained (95% CI=[0.752, 0.963]. Based on this model, the following were obtained: sensitivity=0.76, specificity=0.85, PPV=0.73, and NPV 0.92 (out-of-sample sensitivity=0.89, specificity=0.79, PPV=0.83, and NPV=0.79).

According to some aspects, the method includes comparing a number of triangles with negative values between longitudinal MRI data of a patient at step 118. For example, the processor 610 illustrated in FIG. 6 may compare a number of triangles with negative values between longitudinal MRI data of a patient. The method may include determining the patient having a rate of increase in a number of introverted triangles of greater than 10 triangles per year or a rate of decrease in a number of extroverted triangles of greater than 10 triangles per year has an increased risk of MS and a decreased risk of NMOSD at step 120. For example, the processor 610 illustrated in FIG. 6 may determine that the patient has a rate of increase in a number of introverted triangles of greater than 10 triangles per year or a rate of decrease in a number of extroverted triangles of greater than 10 triangles per year has an increased risk of MS and a decreased risk of NMOSD.

According to some aspects, the method includes determining the patient having an insignificant rate of change of a number of introverted triangles or extroverted triangles has an increased risk of NMOSD and decreased risk of MS at step 122. For example, the processor 610 illustrated in FIG. 6 may determine the patient has an insignificant rate of change of a number of introverted triangles or extroverted triangles has an increased risk of NMOSD and decreased risk of MS.

Such a determination is based on the research that resulted in the present diagnostic technology. The research found that the number of estimated triangles representing an introverted planar contour for an NMOSD subject of average age (40.0 years) and average disease duration (8.9 years) at baseline was 99.2 (95% CI=[86.89, 111.50]). At baseline, patients with MS had 38.75 less triangles representing an introverted planar contour than those with NMOSD (p<0.0001, 95% CI=[−50.56,−26.95]) or less introverted surface characteristics. The rate of change within the MS cohort was found to be significantly greater than in the NMOSD group with an expected increase of 12.72 triangles per year and topography within the region of interest evolving from having less elaborate surface characteristics to more complex features (p=0.0008, 95% CI=[5.60, 19.87]). The rate of change in these triangles between NMOSD and MOGAD patients was not significant (p=0.07). Age (p=0.08), time from symptom onset (p=0.50), treatment at the time of scan (p=0.51), and relapse occurrence between scans (p=0.14) were not found to be statistically significant.

The number of triangles representing an extroverted planar contour for NMOSD subjects of average age (40.0 years) and average disease duration (8.9 years) at baseline was 59.60 (95% CI=[47.22, 72.01]). Patients with MS had approximately 33.01 more triangles representing an extroverted planar contour than NMOSD subjects (p<0.0001, 95% CI=[21.18, 44.83]), or more extroverted surface features. The rate of change in the MS cohort was more dynamic when compared to NMOSD and an expected decrease in triangles representing an extroverted planar contour by 13.38 per year (p=0.0005, 95% CI=[−20.53,−6.20]) with evolution from extroverted characteristics to more complex and introverted surface topography was identified. No difference in such triangles were observed between NMOSD and MOGAD cohorts (p=0.16). Time from symptom onset (p=0.44), treatment at the time of scan (p=0.86), and relapse (p=0.11) was not statistically significant. However, a one standard deviation increase in age (11.15 years) decreases the number of red triangles by 4.28 per year (p=0.02, 95% CI=[−7.82,−0.75]).

According to some aspects, the method includes determining a patient has a presence of a distinct spatial dissemination pattern of introverted triangles extending craniocaudally within a center of the ROI has an increased risk of NMOSD and MOGAD and a decreased risk of MS at step 124. For example, the processor 610 illustrated in FIG. 6 may determine a patient has a presence of a distinct spatial dissemination pattern of introverted triangles extending craniocaudally within a center of the ROI has an increased risk of NMOSD and MOGAD and a decreased risk of MS.

Such a determination is based on the research that resulted in the present diagnostic technology. The research found that the presence of a distinct spatial dissemination pattern of triangles extending craniocaudally within the center of the region of interest was observed in NMOSD patients. The presence of such a finding was significant when comparing NMOSD and MS patients (p<0.0001). Similar features were observed in the MOGAD cohort and when compared to MS patients, a statistically significant difference was present (p<0.0001). No differences in the spatial dissemination pattern of triangles representing introverted planar contours were observed between NMOSD and MOGAD patients (p>0.99), MOGAD HT and LT (p>0.99), and AQP4-IgG positive and AQP4-IgG negative NMOSD (p=0.39).

According to some aspects, the method includes defining a treatment efficacy including at least on of approved therapies and experimental therapies based on defined metrics including at least one of an introverted triangle count, an extroverted triangle count, longitudinal texture measures, temporal profile changes over time, and a spatial dissemination pattern of the introverted triangles. According to some aspects, the method includes defining a treatment's success or failure based on defined metrics including at least one of an introverted triangle count, an extroverted triangle count, longitudinal texture measures, temporal profile changes over time, and a spatial dissemination pattern of the introverted triangles. According to some aspects, the method includes tracking disease behavior clinically and define a natural history of disease or a treatment's success or failure based on defined metrics including at least one of an introverted triangle count, an extroverted triangle count, longitudinal texture measures, temporal profile changes over time, and a spatial dissemination pattern of the introverted triangles. Natural history here means the evaluation of disease behavior in the absence of treatment.

FIG. 2 illustrates a region of interest at the dorsal medulla (yellow circle) along with the corresponding location (white mesh) within 3-dimensional (3D) mesh models (blue) and 3D visualization models containing the region of interest demonstrating the associated planar contours and surface topography.

FIG. 3A illustrates texture characteristics within the region of interest at the dorsal medulla from a patient with NMOSD, MS, and MOGAD demonstrating differences in the number and pattern of triangles with introverted (blue) and extroverted (red) planar contours. A higher number of triangles representing introverted planar contours or more concavity was observed in NMOSD (117) as compared to MOGAD (71) and MS (52). For triangles comprising an extroverted, or more extroverted surface, higher numbers were observed in MS (92) as compared to MOGAD (82) and NMOSD (58). Note in NMOSD and MOGAD that the introverted planar contours span craniocaudally as compared to MS. B. Illustration of key dorsal medullary structures encompassed by the region of interest (blue, red).37 4V=fourth ventricle, E=ependymal and subependymal layer, AP=area postrema, gr=gracile fasciculus, GR=gracile nucleus, SoIDL=dorsal lateral solitary nucleus.

FIG. 3B illustrates an example three-dimensional image 300B of the dorsal aspect of the brainstem and upper cervical spinal cord created using data from two MRI time points. In an example use case of using texture characteristic changes using two MRI time points, regions of selective vulnerability within the central nervous system by race and ethnicity may be identified.

According to some aspects, thresholds may be defined for quantifying change within the brainstem-upper cervical spinal cord. Three-dimensional images of the dorsal aspect of the brainstem and upper cervical spinal cord, for example, may be created using data from two MRI time points. A first color, such as white as shown in FIG. 3B, may represent regions of no change. A second color, such as red as shown in FIG. 3B, may depict areas where tissue volume has decreased between the two MRI time points. A third color, such as blue as shown in FIG. 3B, may represent regions where volume has increased between the two MRI time points. The increase in the intensity of the color may also be associated with higher volume changes within that region. Cutoffs may be defined and used for identifying changes within a given region of interest.

In some cases, the systems and methods disclosed herein may be used to identify changes of interest within a given region of interest, for example, identifying regions of selective vulnerability within the central nervous system by race and ethnicity. Additional details regarding methods and systems for analyzing a central nervous system based on brainstem structural characteristics are discussed in relation to International Application No. PCT/US21/28898 titled “METHODS AND SYSTEMS FOR ANALYZING A CENTRAL NERVOUS SYSTEM BASED ON BRAINSTEM STRUCTURAL CHARACTERISTICS,” which is incorporated herein by reference in its entirety. As discussed in further detail below, identifying changes of interest within a given region of interest can be implemented using three-dimensional conformational characteristics to allow for the identification of where a volume change takes place within a three-dimensional structure. For example, as discussed in International Application No. PCT/US21/28898, differences at the dorsal medulla were identified between those of Black or African American ancestry versus those of European ancestry who have MS. As such, the methods disclosed herein may be used for identifying regions of selective vulnerability within the central nervous system by race and ethnicity.

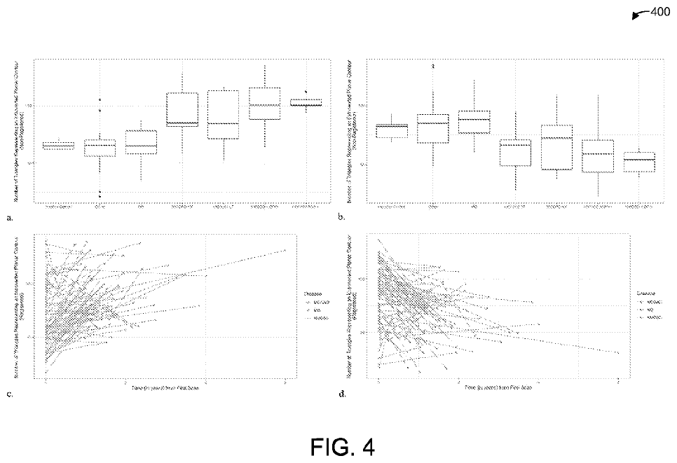

FIG. 4 illustrates box plots and spaghetti plots associated with the research performed that resulted in the present diagnostic technology. The box plots for the number of triangles representing introverted (A) and extroverted (B) planar contours measured cross-sectionally by group and Spaghetti plots of longitudinal measures of triangles representing introverted (C) and extroverted (D) planar contours from registered MRI scans in patients with MOGAD, MS, and NMOSD.

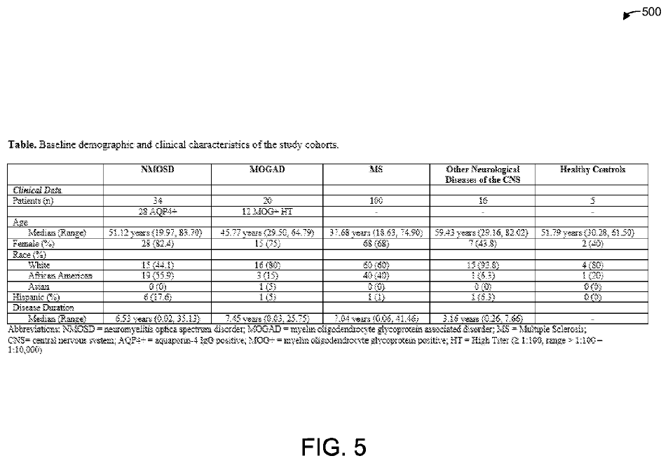

FIG. 5 illustrates a table for baseline demographic and clinical characteristics of the study cohorts, in accordance with some examples. Inclusion criteria were comprised of i) male or female patients >18 years of age with ii) an established diagnosis of relapsing-remitting MS, AQP4-IgG positive or AQP4-IgG negative NMOSD, MOGAD following a comprehensive medical evaluation by fellowship-trained specialists in neuroimmunology, iii) no observable focal lesions within the brainstem, v) lack of exposure to oral or intravenous glucocorticosteroid treatment 90 days prior to MRI, and vi) no change in disease-modifying therapy (DMT) or immunosuppressive treatment within 90 days prior to MRI. Exclusion criteria included i) female patients who were pregnant or lactating, ii) severe claustrophobia, and iii) reduced quality of MRI data limiting the 3D image processing.

The following further describes the research performed that resulted in the present diagnostic technology. All research patients included in this study along with a single healthy control were recruited from The University of Texas Southwestern Medical Center (UT Southwestern) in Dallas, Texas. Other healthy control subjects were recruited from the Dallas-Fort Worth metroplex using posted flyers. This study was approved by the Institutional Review Board at UT Southwestern.

A healthy control group was also included that was comprised of subjects with no history of brain anomalies typical for CNS demyelination based on the observed radiological features and formal imaging interpretations by board certified neuroradiologists and clinical impressions by specialists in MS.

Patients included in the analysis were placed into the following groups based on established diagnostic criteria:
i) MS,
ii) AQP4-IgG positive (AQP4+) NMOSD,
iii) AQP4-IgG negative (AQP4−) NMOSD,
iv) MOGAD,
v) other neurological diseases of the CNS, and
vi) healthy control subjects.

As diagnostic criteria for MOGAD were not available, included subjects were placed into two groups,
i) low-titer (<1:100) and
ii) high-titer (>1:100).

Within the other neurological diseases category, patients with genetically confirmed cerebral autosomal dominant arteriopathy subcortical infarcts and leukoencephalopathy (CADASIL), metachromatic leukodystrophy, and hereditary diffuse leukoencephalopathy with spheroids (HDLS) were included. Antibody confirmed limbic encephalitis, and patients with Alzheimer's Disease, mild cognitive impairment, frontotemporal dementia, and multi-infarct dementia were also included.

All imaging studies were performed at The University of Texas Southwestern Medical Center in Dallas, Texas on 3T MRI scanners (Achieva, Philips Medical Systems, Cleveland, OH) located on campus using a 32-channel phased array head coil for reception and the built-in scanner body coil for transmission under a standardized protocol. Each MRI study included scout localizers, 3D high-resolution magnetization prepared rapid gradient echo (MPRAGE) T1-weighted isotropic (1.1×1.1×1.1 mm3, TE/TR/TI=3.7/8.1/864 ms, flip angle 12 degrees, 256×220×170 mm3 FOV, number of excitations (NEX)=1, 170 slices, duration: 4:11 min), 3D fluid-attenuated inversion recovery (FLAIR) (1.1×1.1×1.1 mm3, TE/TR/TI=350/4800/1600 ms, flip angle 90 degrees, 250×250×180 mm3 FOV, NEX=1, 163 slices, duration: 5:02 min) and 3D T2-weighted turbo spin echo sequence acquired in the sagittal plane (1.0×1.0×1.0 mm3, TE/TR/TI=229/2500/1600 ms, flip angle 90 degrees, 250×250×180 mm3 FOV, NEX=1, 164 slices, duration: 4:33 min).

The sphere used as the ROI was identified as superior to 2D rectangles, 2D squares, 3D cubes, 3D prisms, 2D circular/ovoid selections, 3D cones, 3D tubular structures, which all failed. The sphere was able to create the selection of interest given the anatomy present at the dorsal medulla and the pattern of disease involvement observed in NMOSD and MOG in this location, and sizing was also critical, as FIG. 3 demonstrates. The selection should be of a certain size because the measures of the presence or absence of introverted/extroverted triangles will be affected by the inclusion of surrounding triangles, given that a single triangle has no bend or curve in it. The incorporation of surrounding triangles, taken together, can have surface curvatures, however.

All post-processing of acquired MRI imaging studies was performed without knowledge of demographic data, clinical history, current or past treatments, or disease duration. For longitudinal data included in this study, the 3D T1-weighted MRI sequences were initially registered based on structural positioning and intensity using an in-house developed software package previously utilized in prior studies, Med-IP.8-11 MRI studies were aligned using the Insight Toolkit (ITK) (version 5.1.1; Kitware Clifton Park, N.Y., U.S.A.) multi-resolution rigid registration with Mattes Mutual Information Metric. Histogram matching of intensities involving regions of interest through linear transforms and ordered correspondence on a set of match points computed from the quantiles of each histogram was performed to ensure proper intensity alignment. For patients and healthy controls with a single MRI timepoint, the segmentation was directly performed using the MPRAGE sequence.

All analyses were performed in R (version 4.0.5). All plots were generated using the ggplot2 package. Linear mixed effects models were implemented using the ImerTest package. Optimal cut-offs discriminating NMOSD and MS were determined using the OptimalCutpoint package.

Comparisons of absolute introverted planar and extroverted planar triangle counts between diagnoses were performed using two-sample t-tests if normality assumptions were verified and Mann-Whitney U test otherwise. Determination of appropriate cut-offs was performed by determining the number of triangles representing an introverted (introverted) or extroverted (extroverted) planar contour present within the region of interest in NMOSD subjects with the greatest Youden's J statistic (or maximum sensitivity and specificity summation) in a training set containing 70% of the data. The resulting 30% of data were used as the training set to obtain a measure of out-of-sample measurements of sensitivity, specificity, positive predictive value (PPV), and negative predictive value (NPV).

The in-sample area under the curve (AUC), sensitivity, specificity, PPV, and NPV corresponded to the training set evaluated at the optimal cutoff and the out-of-sample sensitivity, specificity, PPV, and NPV corresponded to the results of the testing set evaluated at the optimal cutoff. Comparisons of the proportion of patients exhibiting distinct craniocaudal spatial distribution patterns of triangles representing introverted planar contours by diagnosis was performed using Fisher's Exact test. Lastly, linear mixed effects models were fit to the longitudinal data with covariates including diagnosis, time from first scan, centered-and-scaled time from symptom onset, centered-and-scaled age, an indicator variable corresponding to one if a subject was on treatment at the time of the scan and zero otherwise, and an indicator variable corresponding to one if the subject experienced a relapse between scans and zero otherwise. A subject-specific random intercept was included in the linear mixed effects model.

A total of 114 NMOSD and 75 MOGAD patients were identified in our center from 2017-2021, with 34 (29.8%) NMOSD and 20 (26.7%) MOGAD patients having at least one 3D MRI time point. All patients with at least one 3D MRI time point were included in the analysis. The study cohort was comprised of 34 NMOSD patients (28 female; median age=51.12 years (y), range (19.97, 83.70) with a median disease duration of 6.53y (0.02, 35.13)), 20 MOGAD (15 female; median age=45.77y (29.50, 64.79) with a median disease duration of 7.45y (0.03, 25.75)), 100 MS (68 female; median age=37.68y (18.63, 74.90) having a median disease duration of 7.04y (0.06, 41.46)), 16 patients with other neurological diseases of the CNS (15 female; median age=59.43y (29.16, 82.02) with a median disease duration of 3.16 years (0.26, 7.66)).

The other neurological diseases of the CNS group was comprised of well clinically characterized patients from our university with genetically confirmed cerebral autosomal dominant arteriopathy subcortical infarcts and leukoencephalopathy (CADASIL) (2), metachromatic leukodystrophy (1), hereditary diffuse leukoencephalopathy with spheroids (HDLS) (1), and mitochondrial disease (1). Others included patients with confirmed limbic encephalitis LGI1antibodies (3), Alzheimer's Disease (5), mild cognitive impairment (1), frontotemporal dementia (1), and multi-infarct dementia (1). In addition, 5 healthy controls were included (2 female; 51.79y (30.28, 61.50)). All patients within the study cohort had serological testing performed at Mayo Clinic Laboratories, Rochester, Minnesota with testing dates for AQP4-IgG ranging from 2012 to 2021 and MOG-IgG from 2018 to 2021.

The early recognition and accurate diagnosis of neurodegenerative diseases is critical in ensuring rapid treatment implementation aimed at reducing irreversible damage and permanent neurological disability. In this study, we demonstrated that cross-sectional and longitudinal dorsal medullary surface texture measures differ between NMOSD and MS patients. Near the time of diagnosis, NMOSD patients appeared to have dorsal medullary surface texture characteristics at the region of interest that were more introverted and complex than in MS patients. Interestingly, over time the changes in surface texture in MS patients appeared more dynamic in evolution as compared to those with NMOSD, transitioning to a structure with greater general surface complexity. Key differences in the spatial distribution of triangles representing introverted planar contours between NMOSD and MOGAD, when compared to MS, were also seen. The presented approach provides insights into key surface textural differences and spatial dissemination characteristics of triangles that relate to surface contour within a distinct region of the CNS and may be of value as an additional metric in the clinical evaluation of patients with suspected NMOSD or MS. Additional insights may be gained regarding the impact of this area in those patients diagnosed with AQP4-IgG negative NMOSD and MOGAD.

Volumetric data related to the study of NMOSD has contributed to our understanding of the impact of NMOSD on cortical grey matter, deep grey matter, hippocampus, brainstem, and brain volume measures. The incorporation of texture adds an alternative form of information, providing information related to the integrity of tissue in regions where volumetric analyses cannot easily be performed. The approach also enables the identification of the spatial pattern of change within a defined structure that has decreased in volume. By studying the number of introverted and extroverted triangles cross-sectionally, we were able to identify distinct patterns of change between NMOSD and MS cohorts. Triangles representing introverted or extroverted planar contours are present in normal anatomical structures but change in response to tissue injury or secondary neurodegenerative change. The identification of variations in the pattern of introverted and extroverted planar contours inform on microscopic structural changes in the dorsal medulla that reveal the presence of atrophy in select regions and thus, greater surface complexities. The differences observed in the number of introverted and extroverted triangles are consistent with the underlying pathophysiology of NMOSD and MS.

Central to the pathophysiology of NMOSD are AQP4-IgG antibodies that bind selectively to the mercurial-insensitive water sensitive transporting proteins. This interaction results primarily in in optic nerve and spinal cord injury although brain regions of high AQP4 expression may also be affected. The approach of focusing on changes in surface texture near the region of the area postrema is strategic given the enrichment of AQP4 channels concentrated in astrocytic foot processes in that location and resulting selective vulnerability to antibody mediated injury. The observation of introverted planar contours spanning craniocaudally in this region of interest in NMOSD but not in MS further provides supporting evidence for the mechanism and target of injury in accordance with the relevant anatomy. The lower medulla also serves as an ideal region for study given the defined anatomical boundaries with low inter-individual differences and decreased propensity for measures to be impacted by variations in anatomy. This region is also highly accessible without the need for a dedicated sequence for acquisition. Prior histopathological data in the study of this region also support a reduction in tissue density in the setting of non-obvious axonal pathology or myelin loss, findings supportive of non-destructive change that allow for tissue topography to be effectively quantified.

Longitudinal texture measures were applied in patients with two or more MRI time points to evaluate the consistency of texture measures over time and to explore temporal profile changes that may inform on disease state. Within a modest group of NMOSD patients with longitudinal MRI studies, we identified significantly lower rates of change in the number of triangles representing introverted planar contours at the region of interest in the dorsal medulla when compared to MS patients. This finding appears to be consistent with the lack of typical disease progression seen in NMOSD and observed changes over time may be reflective of secondary changes resulting from earlier injury. Whether further elevations are informative of ongoing disease activity is currently unclear. In contrast, the change in tissue topography within the region of interest was more dynamic in the MS group with transitions to more complex surfaces observed. This finding may be reflective of the more destructive nature of MS with ongoing early neurodegeneration separate from focal inflammatory lesions.

Individuals fulfilling criteria for AQP4-IgG negative NMOSD demonstrating seropositivity for MOGAD are well described within the scientific literature. Although key differences in the clinical presentations of these conditions exist with MOG-IgG antibodies patients having spatial dissemination differences in lesions with optic nerve lesions distal to the chiasm, the presence of a sagittal line within the cervical spine and distinct involvement of grey matter, involvement of the conus medullaris, and better functional recovery, a difference in dorsal medullary texture was not identified when compared to NMOSD. These results suggest that MOGAD and NMOSD are more homologous in regards to involvement within the region of interest studied as compared to MS and that the area postrema and nearby regions may be selectively vulnerable to non-inflammatory injury in these conditions. As MOG is only present in modest amounts within myelin and that preferential loss is not observed histopathologically, disruption of the organization of thin filaments and microtubule cytoskeleton may serve as one of the reasons for the observed changes in surface texture. These data are consistent with the clinical observation of intractable nausea, vomiting, or hiccups in patients with MOGAD and NMOSD and our texture findings suggest a sensitive measure for documenting involvement below the threshold of conventional MRI and clinical symptoms. Whether certain MOGAD texture profiles are associated with more aggressive clinical courses still needs to be determined.

The approach of the research was to incorporate a dorsal medullary surface texture measure, obtained via a commonly acquired sequence in research and clinical practice, provides a number of other additional benefits. The availability of the results from the first MRI scan may be immediate for healthcare providers. The texture metric acquired during follow-up imaging studies could also serve as a complement to the medical workup while awaiting the results from repeat antibody testing performed after an initial negative result. In addition, the measurement of dorsal medullary texture appears to be stable across MRI scanners utilizing the same protocol and the longitudinal data appear promising as a measure for studying long-term tissue changes related to disease.

The number of subjects with NMOSD included in this study was modest and therefore the results should be verified in a larger dataset. How surface texture changes evolve over a longer period of time and the impact of the available approved treatments with varying mechanisms of action remain uncertain. The data suggest that the measures are stable within both white and non-white groups, however measures from other ethnicities throughout the world remain unknown. In addition, the contribution of sex to the measures are also unknown. As the criteria for MOGAD are currently limited, identified subjects were included based on known clinical features and stratified based on MOG-IgG titer. Therefore, future studies could potentially reveal differences or stronger associations in dorsal medullary texture metrics between NMOSD and MOGAD.

Studies involving a larger, more diverse group of individuals with NMOSD are needed. The data presented here support the value of imaging approaches specifically targeting regions of the CNS at increased propensity for injury. In addition, the future incorporation of structural measures beyond volumetrics, such as texture, may allow for the generation of new radiological measures in the field of neuroimmunology. If reliable, these measures may ultimately result in the expansion of clinical characteristics along with revisions to definitions of disease regression, stability, and progression.

FIG. 6 shows an example of a computing system in accordance with some aspects of the present disclosure. FIG. 6 shows an example of computing system 600, which can be for example any computing device making up the core network 230, or any component thereof in which the components of the system are in communication with each other using connection 605. Connection 605 can be a physical connection via a bus, or a direct connection into one or more processors 610, such as in a chipset architecture. Connection 605 can also be a virtual connection, networked connection, or logical connection.

In some embodiments computing system 600 is a distributed system in which the functions described in this disclosure can be distributed within a datacenter, multiple datacenters, a peer network, etc. In some embodiments, one or more of the described system components represents many such components each performing some or all of the function for which the component is described. In some embodiments, the components can be physical or virtual devices.

Example system 600 includes at least one processing unit (CPU or processor) 610 and connection 605 that couples various system components including system memory 615, such as read only memory (ROM) 620 and random access memory (RAM) 625 to one or more processors 610. Computing system 600 can include a cache of high-speed memory 612 connected directly with, in close proximity to, or integrated as part of process one or more processors 610.

One or more processors 610 can include any general purpose processor and a hardware service or software service, such as services 632, 634, and 636 stored in storage device 630, configured to control processor 610 as well as a special-purpose processor where software instructions are incorporated into the actual processor design. One or more processors 610 may essentially be a completely self-contained computing system, containing multiple cores or processors, a bus, memory controller, cache, etc. A multi-core processor may be symmetric or asymmetric.

To enable user interaction, computing system 600 includes an input device 645, which can represent any number of input mechanisms, such as a microphone for speech, a touch-sensitive screen for gesture or graphical input, keyboard, mouse, motion input, speech, etc. Computing system 600 can also include output device 635, which can be one or more of a number of output mechanisms known to those of skill in the art. In some instances, multimodal systems can enable a user to provide multiple types of input/output to communicate with computing system 600. Computing system 600 can include communications interface 640, which can generally govern and manage the user input and system output. There is no restriction on operating on any particular hardware arrangement and therefore the basic features here may easily be substituted for improved hardware or firmware arrangements as they are developed.

Storage device 630 can be a non-volatile memory device and can be a hard disk or other types of computer readable media which can store data that are accessible by a computer, such as magnetic cassettes, flash memory cards, solid state memory devices, digital versatile disks, cartridges, random access memories (RAMs), read only memory (ROM), and/or some combination of these devices.

The storage device 630 can include software services, servers, services, etc., that when the code that defines such software is executed by the one or more processors 610, it causes the system to perform a function. In some embodiments, a hardware service that performs a particular function can include the software component stored in a computer-readable medium in connection with the necessary hardware components, such as processor 610, connection 605, output device 635, etc., to carry out the function.

For clarity of explanation, in some instances the present technology may be presented as including individual functional blocks including functional blocks comprising devices, device components, steps or routines in a method embodied in software, or combinations of hardware and software.

Any of the steps, operations, functions, or processes described herein may be performed or implemented by a combination of hardware and software services or services, alone or in combination with other devices. In some embodiments, a service can be software that resides in memory of a client device and/or one or more servers of a content management system and perform one or more functions when a processor executes the software associated with the service. In some embodiments, a service is a program, or a collection of programs that carry out a specific function. In some embodiments, a service can be considered a server. The memory can be a non-transitory computer-readable medium.

In some embodiments the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Methods according to the above-described examples can be implemented using computer-executable instructions that are stored or otherwise available from computer readable media. Such instructions can comprise, for example, instructions and data which cause or otherwise configure a general purpose computer, special purpose computer, or special purpose processing device to perform a certain function or group of functions. Portions of computer resources used can be accessible over a network. The computer executable instructions may be, for example, binaries, intermediate format instructions such as assembly language, firmware, or source code. Examples of computer-readable media that may be used to store instructions, information used, and/or information created during methods according to described examples include magnetic or optical disks, solid state memory devices, flash memory, USB devices provided with non-volatile memory, networked storage devices, and so on.

Devices implementing methods according to these disclosures can comprise hardware, firmware and/or software, and can take any of a variety of form factors. Typical examples of such form factors include servers, laptops, smart phones, small form factor personal computers, personal digital assistants, and so on. Functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are means for providing the functions described in these disclosures.

Although a variety of examples and other information was used to explain aspects within the scope of the appended claims, no limitation of the claims should be implied based on particular features or arrangements in such examples, as one of ordinary skill would be able to use these examples to derive a wide variety of implementations. Further and although some subject matter may have been described in language specific to examples of structural features and/or method steps, it is to be understood that the subject matter defined in the appended claims is not necessarily limited to these described features or acts. For example, such functionality can be distributed differently or performed in components other than those identified herein. Rather, the described features and steps are disclosed as examples of components of systems and methods within the scope of the appended claims.

Claim language reciting “at least one of” a set indicates that one member of the set or multiple members of the set satisfy the claim. For example, claim language reciting “at least one of A and B” means A, B, or A and B.

1. A computer-implemented method to determine an increased likelihood of a neurological disorder based on a dorsal surface texture of a medulla oblongata, the computer-implemented method comprising:
selecting, using a computational topography software, a region of interest (ROI) within a diameter ring at a lower dorsal posterior medulla encompassing a region of a clava;
analyzing, using the computational topography software, surface complexity of the ROI using a metric maximum curvature analysis that provides a local maximum curvature on discretized triangular mesh surface representations; and
determining between introverted triangles and extroverted triangles and calculating a first number of the introverted triangles and a second number of the extroverted triangles in the ROI.

2. The computer-implemented method of claim 1, wherein the determining between the introverted triangles and the extroverted triangles includes assigning a curvature value to each triangle in a context of neighboring triangles, wherein negative triangle values indicated a more introverted surface and positive values indicated a more extroverted surface.

3. The computer-implemented method of claim 1, further comprising:
determining a patient having more than 89 introverted triangles or having less than 70 extroverted triangles has an increased risk of NMOSD.

4. The computer-implemented method of claim 1, further comprising:
determining if an individual has a myelin oligodendrocyte glycoprotein (MOG) IgG titer greater than 1:100 or less than 1:100 from a blood test; and
determining a patient having more than 89 introverted triangles or having less than 70 extroverted triangles has an increased risk of myelin oligodendrocyte glycoprotein associated disorder (MOGAD).

5. The computer-implemented method of claim 1, further comprising:
computing, using the computational topography software, a curative measure for triangles within the ROI using a least-square fitting technique encompassing computational measure at each triangle node to unify triangle size.

6. The computer-implemented method of claim 1, further comprising:
using non-registered or registered 3D T1-weighted magnetic resonance imaging (MRI) sequences for tracking position and shape changes of structures; and
identifying the ROI from a superior colliculus of a midbrain to a caudal end of the medulla oblongata from non-contrast-enhanced 3D isotropic T1-weighted magnetic resonance imaging (MRI) sequences.

7. The computer-implemented method of claim 1, further comprising:
comparing a number of triangles with negative values within ROIs of a plurality of patients, wherein a higher number of triangles with negative values informed on more introverted features within the region of interest.

8. The computer-implemented method of claim 1, further comprising:
comparing a number of triangles with negative values between longitudinal MRI data of a patient; and
determining the patient having an insignificant rate of change of a number of introverted triangles or extroverted triangles has an increased risk of NMOSD and decreased risk of MS.

9. The computer-implemented method of claim 1, further comprising:
comparing a number of triangles with negative values between longitudinal MRI data of a patient; and
determining the patient having a rate of increase in a number of introverted triangles of greater than 10 triangles per year or a rate of decrease in a number of extroverted triangles of greater than 10 triangles per year has an increased risk of MS and a decreased risk of NMOSD.

10. The computer-implemented method of claim 1, further comprising:
determining a patient has a presence of a distinct spatial dissemination pattern of introverted triangles extending craniocaudally within a center of the ROI has an increased risk of NMOSD and MOGAD and a decreased risk of MS.

11. A system to determine an increased likelihood of a neurological disorder based on a dorsal surface texture of a medulla oblongata, the system comprising:
a storage configured to store instructions; and
a processor configured to execute the instructions and cause the processor to:
select, use a computational topography software, a region of interest (ROI) within a diameter ring at a lower dorsal posterior medulla encompassing a region of a clava,
analyze, use the computational topography software, surface complexity of the ROI using a metric maximum curvature analysis that provides a local maximum curvature on discretized triangular mesh surface representations, and
determine between introverted triangles and extroverted triangles and calculate a first number of the introverted triangles and a second number of the extroverted triangles in the ROI.

12. The system of claim 11, wherein the determining between the introverted and the extroverted triangles further causes the processor to:
assign a curvature value to each triangle in a context of neighboring triangles, wherein negative triangle values indicated a more introverted surface and positive values indicated a more extroverted surface.

13. The system of claim 11, wherein the processor is configured to execute the instructions and cause the processor to:
determine a patient having more than 89 introverted triangles or have less than 70 extroverted triangles has an increased risk of NMOSD.

14. The system of claim 11, wherein the processor is configured to execute the instructions and cause the processor to:
determine an individual has a myelin oligodendrocyte glycoprotein (MOG) IgG titer greater than 1:100 or less than 1:100 for; and
determine a patient having more than 89 introverted triangles or have less than 70 extroverted triangles has an increased risk of myelin oligodendrocyte glycoprotein associated disorder (MOGAD).

15. The system of claim 11, wherein the processor is configured to execute the instructions and cause the processor to:
compute, use the computational topography software, a curative measure for triangles within the ROI using a least-square fitting technique encompassing computational measure at each triangle node to unify triangle size.

16. The system of claim 11, wherein the processor is configured to execute the instructions and cause the processor to:
use non-registered or registered 3D T1-weighted magnetic resonance imaging (MRI) sequences for track position and shape changes of structures; and
identify the ROI from a superior colliculus of a midbrain to a caudal end of the medulla oblongata from non-contrast-enhanced 3D isotropic T1-weighted magnetic resonance imaging (MRI) sequences.

17. A non-transitory computer-readable medium comprising instructions, the instructions, when executed by a computing system, cause the computing system to:
select, use a computational topography software, a region of interest (ROI) within a diameter ring at a lower dorsal posterior medulla encompassing a region of a clava;
analyze, use the computational topography software, surface complexity of the ROI using a metric maximum curvature analysis that provides a local maximum curvature on discretized triangular mesh surface representations; and
determine between introverted triangles and extroverted triangles and calculate a first number of the introverted triangles and a second number of the extroverted triangles in the ROI.

18. The non-transitory computer-readable medium of claim 17, wherein the non-transitory computer-readable further comprises instructions that, when executed by the computing system, cause the computing system to:
compare a number of triangles with negative values within ROIs of a plurality of patients, wherein a higher number of triangles with negative values informed on more introverted features within the region of interest.

19. The non-transitory computer-readable of claim 17, wherein the non-transitory computer-readable further comprises instructions that, when executed by the computing system, cause the computing system to:
compare a number of triangles with negative values between longitudinal MRI data of a patient; and
determine that the patient has an insignificant rate of change of a number of introverted triangles or extroverted triangles has an increased risk of NMOSD and decreased risk of MS.

20. The non-transitory computer-readable of claim 17, wherein the non-transitory computer-readable further comprises instructions that, when executed by the computing system, cause the computing system to:
compare a number of triangles with negative values between longitudinal MRI data of a patient; and
determine that the patient has a rate of increase in a number of introverted triangles of greater than 10 triangles per year or a rate of decrease in a number of extroverted triangles of greater than 10 triangles per year has an increased risk of MS and a decreased risk of NMOSD.

21. (canceled)

22. (canceled)

23. (canceled)
